(19) United States
(12) Patent Application Publication
(10) Pub. No.: US 2025/0125059 A1
(43) Pub. Date: Apr. 17, 2025

(54) SYSTEMS AND METHODS FOR IMPLEMENTING INTERACTIVE GRAPHICAL USER INTERFACES FOR ACCELERATED VIRTUAL SIMULATIONS AND MANIPULATION OF CLINICAL TRIAL DATA FOR GENERATING CLINICAL TRIAL-RELATED INTELLIGENCE

(71) Applicant: Sleuth Insights, Inc., San Francisco, CA (US)

(72) Inventors: Andrew Pannu, Alamo, CA (US); Michael Kim, Walnut Creek, CA (US); Sean Byrnes, Piedmont, CA (US)

(73) Assignee: Sleuth Insights, Inc., San Francisco, CA (US)

(21) Appl. No.: 18/914,848

(22) Filed: Oct. 14, 2024

Related U.S. Application Data
(60) Provisional application No. 63/543,910, filed on Oct. 12, 2023.

(51) Int. Cl. G16H 50/50; G16H 10/20

(52) U.S. Cl. CPC G16H 50/50; G16H 10/20

(57) ABSTRACT

A system and method for implementing interactive graphical user interfaces (GUIs) for accelerated integration and manipulation of clinical trial data for generating clinical trial-related intelligence may include obtaining a clinical trial data corpus associated with candidate clinical trials; deriving a statistical model for each of the candidate clinical trials; computing a composite statistical model from the statistical models; generating a synthetic statistical model for a target clinical trial by adapting the composite statistical model; initializing a virtual simulation computing system with simulation parameters linked to the synthetic statistical model; executing, by the virtual simulation computing system, virtual simulations using the simulation parameters; generating clinical trial intelligence data including a graphical simulation artifact from the virtual simulations; providing the graphical simulation artifact to a first display section of an interactive GUI; and providing editable user interface input elements to a second display section of the interactive GUI for configuring simulation parameters.

### CROSS-REFERENCE TO RELATED APPLICATIONS

This application claims the benefit of U.S. Provisional Application No. 63/543,910, filed 12 Oct. 2024, which is incorporated herein in its entirety by this reference.

### TECHNICAL FIELD

This invention relates generally to a graphic user interface (GUI) and more specifically, to a new and useful system and method for implementing graphical user interfaces for displaying and manipulating clinical trial-related intelligence data.

### BACKGROUND

Clinical trials are essential for evaluating the safety and efficacy of new medical treatments, such as pharmaceuticals or medical devices. However, the process of conducting a clinical trial is time-consuming and prone to uncertainty. Historically, researchers have sought to mitigate these challenges by assessing the chemical properties of a drug and using simulations to predict how it will behave in a human body. While such approaches offer some value, they suffer from limitations in the inaccuracy in predicting trial outcomes for diverse patient populations.

Additionally, previous systems that may rely on information from a clinical trial to predict outcomes of future trials may be limited in their accuracy. Specifically, information from a clinical trial may not be directly applicable to another trial whose outcome is yet to be determined, especially when key variables between the two trials differ significantly. Moreover, these prior systems fail to allow users to interactively adjust models based on the specific variables of the new trial, thereby limiting a quality of their predictions.

By contrast, the techniques and systems described in the present application provide an interactive and dynamic graphical user interface (GUI) that enables users to adjust clinical trial models in real-time. This real-time configurability allows users to account for variables unique to the new trial, resulting in more accurate and reliable predictions of clinical trial outcomes.

### BRIEF SUMMARY OF THE INVENTION(S)

In some embodiments, a computer-implemented method may include: at a clinical intelligence service: obtaining, via a computer network from one or more sources of digital data, a corpus of historical clinical trial data associated with each of a plurality of candidate clinical trials; deriving a statistical model for each of the plurality of candidate clinical trials based on the corpus of historical clinical trial data associated with each of the plurality of candidate clinical trials; computing a composite statistical model based on calculating a weighted average of a combination of the statistical model for each of the plurality of candidate clinical trials; generating, by one or more computer processors, a synthetic statistical model for a target clinical trial based on adapting the composite statistical model using a plurality of distinct variable values extracted from data associated with a configuration of the target clinical trial; initializing a virtual simulation computing system with a plurality of simulation parameters including a representation of the synthetic statistical model and a set of input values for a plurality of variables associated with the synthetic statistical model; executing, by the virtual simulation computing system, a plurality of virtual simulations based at least on the initialization of the virtual simulation computing system with the plurality of simulation parameters; generating, by the one or more computer processors, clinical trial intelligence data based on the execution of the plurality of virtual simulations, wherein the clinical trial intelligence data includes one or more graphical simulation artifacts; and providing to at least a first display section of an interactive simulation graphical user interface (GUI), the one or more graphical simulation artifacts and at least a second display section of the interactive simulation GUI a set of editable user interface input elements that, when manipulated, configure or reconfigure one or more of the plurality of simulation parameters thereby enabling a re-execution of a succeeding plurality of virtual simulations and a real-time adaptation of the one or more graphical simulation artifacts based on the re-execution of the succeeding plurality of virtual simulations.

In some embodiments, calculating the weight average includes: setting a respective weight for each candidate trial of the plurality of candidate clinical trials based at least in part on a population size associated with the candidate trial or a similarity of one or more variables associated with the candidate trial to the plurality of distinct variable values extracted from the data associated with the configuration of the target clinical trial, wherein computing the composite statistical model includes applying each respective weight against a respective statistical model for the candidate trial to generate a weighted statistical model; and combining each weighted statistical model to generate the composite statistical model.

In some embodiments, the one or more variables each include a characteristic of a population, a characteristic of a drug, a design characteristic of a clinical trial, or a characteristic of a disease.

In some embodiments, the computer-implemented method further includes: the clinical intelligence service identifying the plurality of candidate clinical trials, wherein identifying the plurality of candidate clinical trials includes utilizing a machine learning model, the machine learning model performing the steps of providing the corpus of historical clinical trial data to the machine learning model, the machine learning model specifically trained to infer confidence values for clinical trials corresponding to a level of similarity between the clinical trials and the target clinical trial; and applying the confidence values to a minimum confidence value threshold to identify the plurality of candidate clinical trials.

In some embodiments, deriving the statistical model for each of the plurality of candidate clinical trials includes: extracting, from the corpus of historical clinical trial data, outcome data for a plurality of samples in the candidate clinical trial, wherein the outcome data includes an outcome for each sample in the plurality of samples; and executing, by the one or more computer processors, a regression on the set of samples to generate a probability distribution for the candidate clinical trial corresponding to the outcome data.

In some embodiments, generating the synthetic statistical model includes: extracting the plurality of distinct variable values from the data associated with the configuration of the target clinical trial data; and transforming the composite statistical model into the synthetic statistical model based at least in part on applying the plurality of distinct variable values to the composite statistical model.

In some embodiments, initializing the virtual simulation computing system with the plurality of simulation parameters includes: loading the representation of the synthetic statistical model and the set of input values for the plurality of variables associated with the synthetic statistical model into a memory of the virtual simulation computing system, wherein the representation of the synthetic statistical model and the set of input values for the plurality of variables associated with the synthetic statistical mode are retrieved from the memory for executing the plurality of virtual simulations.

In some embodiments, the set of editable user interface input elements configure or reconfigure the one or more of the plurality of simulation parameters by triggering the clinical intelligence service to add a candidate clinical trial to the plurality of candidate clinical trials or to remove a candidate clinical trial for re-computation of the composite statistical model prior to re-execution of the succeeding plurality of virtual simulations.

In some embodiments, the set of editable user interface input elements configure or reconfigure the one or more of the plurality of simulation parameters by triggering the clinical intelligence service to update at least one of the plurality of distinct variable values associated with the configuration of the target clinical trial for re-generating the synthetic statistical model prior to re-execution of the succeeding plurality of virtual simulations.

In some embodiments, the plurality of simulation parameters includes the plurality of candidate trials, wherein the set of editable user interface input elements configure the plurality of simulation parameters via selection of the plurality of candidate trials, and wherein initializing the virtual simulation computing system with the plurality of simulation parameters includes loading the selected plurality of candidates into a memory of the virtual simulation computing system, wherein the selected plurality of candidates are retrieved from the memory for computing or re-computing the composite statistical model.

In some embodiments, the computer-implemented method further includes the clinical intelligence service: obtaining, via the computer network from one or more second sources of digital data, a second corpus of historical clinical trial data associated with a second plurality of candidate clinical trials; deriving a statistical model for each of the second plurality of candidate clinical trials based on the second corpus of historical clinical trial data associated with each of the second plurality of candidate clinical trials; computing a second composite statistical model based on calculating a weighted average of a combination of the statistical model for each of the second plurality of candidate clinical trials; generating, by the one or more computer processors, a second synthetic statistical model for the target clinical trial based on adapting the second composite statistical model using a second plurality of distinct variable values extracted from the data associated with the configuration of the target clinical trial; initializing the virtual simulation computing system with a second plurality of simulation parameters including a representation of the second synthetic statistical model and a set of input values for a plurality of variables associated with the second synthetic statistical model; and executing, by the virtual simulation computing system, a second plurality of virtual simulations based at least on the initialization of the virtual simulation computing system with the second plurality of simulation parameters, wherein the one or more graphical simulation artifact of the generated clinical trial intelligence data is based at least in part on executing the second plurality of virtual simulations.

In some embodiments, the plurality of simulation parameters corresponds to an intervention trial and the second plurality of simulation parameters corresponds to a comparator trial.

In some embodiments, the plurality of distinct variable values each include a population size, a characteristic of a drug, a design characteristic of a clinical trial, or a characteristic of a disease.

In some embodiments, a computer-program product embodied in a non-transitory machine-readable storage medium storing computer instructions that, when executed by one or more computer processors, perform operations including: a clinical intelligence service obtaining, via a computer network from one or more sources of digital data, a corpus of historical clinical trial data associated with each of a plurality of candidate clinical trials; deriving a statistical model for each of the plurality of candidate clinical trials based on the corpus of historical clinical trial data associated with each of the plurality of candidate clinical trials; computing a composite statistical model based on calculating a weighted average of a combination of the statistical model for each of the plurality of candidate clinical trials; generating, by the one or more computer processors, a synthetic statistical model for a target clinical trial based on adapting the composite statistical model using a plurality of distinct variable values extracted from data associated with a configuration of the target clinical trial; initializing a virtual simulation computing system with a plurality of simulation parameters including a representation of the synthetic statistical model and a set of input values for a plurality of variables associated with the synthetic statistical model; executing, by the virtual simulation computing system, a plurality of virtual simulations based at least on the initialization of the virtual simulation computing system with the plurality of simulation parameters; generating, by the one or more computer processors, clinical trial intelligence data based on the execution of the plurality of virtual simulations, wherein the clinical trial intelligence data includes one or more graphical simulation artifacts; and providing to at least a first display section of an interactive simulation graphical user interface (GUI), the one or more graphical simulation artifacts, and at least a second display section of the interactive simulation GUI a set of editable user interface input elements that, when manipulated, configure or reconfigure one or more of the plurality of simulation parameters thereby enabling a re-execution of a succeeding plurality of virtual simulations and a real-time adaptation of the one or more graphical simulation artifacts based on the re-execution of the succeeding plurality of virtual simulations.

In some embodiments, the operations to calculate the weighted average include: setting a respective weight for each candidate trial of the plurality of candidate clinical trials based at least in part on a population size associated with the candidate trial or a similarity of one or more variables associated with the candidate trial to the plurality of distinct variable values extracted from the data associated with the configuration of the target clinical trial, wherein the operations to compute the composite statistical model include applying each respective weight against a respective statistical model for the candidate trial to generate a weighted statistical model and combining each weighted statistical model to generate the composite statistical model.

In some embodiments, the one or more variables each include a characteristic of a population, a characteristic of a drug, a design characteristic of a clinical trial, or a characteristic of a disease.

In some embodiments, the operations further include: identifying the plurality of candidate clinical trials, wherein identifying the plurality of candidate clinical trials includes utilizing a machine learning model, the machine learning model performing the steps of providing the corpus of historical clinical trial data to the machine learning model, the machine learning model specifically trained to infer confidence values for clinical trials corresponding to a level of similarity between the clinical trials and the target clinical trial and applying the confidence values to a minimum confidence value threshold to identify the plurality of candidate clinical trials.

In some embodiments, a computer-implemented system may include: one or more processors; a memory; a computer-readable medium operably coupled to the one or more computer processors, the computer-readable medium having computer-readable instructions stored thereon that, when executed by the one or more computer processors, cause a computing device to perform operations including: a clinical intelligence service obtaining, via a computer network from one or more sources of digital data, a corpus of historical clinical trial data associated with each of a plurality of candidate clinical trials; deriving a statistical model for each of the plurality of candidate clinical trials based on the corpus of historical clinical trial data associated with each of the plurality of candidate clinical trials; computing a composite statistical model based on calculating a weighted average of a combination of the statistical model for each of the plurality of candidate clinical trials; generating, by the one or more computer processors, a synthetic statistical model for a target clinical trial based on adapting the composite statistical model using a plurality of distinct variable values extracted from data associated with a configuration of the target clinical trial; initializing a virtual simulation computing system with a plurality of simulation parameters including a representation of the synthetic statistical model and a set of input values for a plurality of variables associated with the synthetic statistical model; executing, by the virtual simulation computing system, a plurality of virtual simulations based at least on the initialization of the virtual simulation computing system with the plurality of simulation parameters; generating, by the one or more computer processors, clinical trial intelligence data based on the execution of the plurality of virtual simulations, wherein the clinical trial intelligence data includes one or more graphical simulation artifacts; and providing to at least a first display section of an interactive simulation graphical user interface (GUI), the one or more graphical simulation artifacts and at least a second display section of the interactive simulation GUI a set of editable user interface input elements that, when manipulated, configure or reconfigure one or more of the plurality of simulation parameters thereby enabling a re-execution of a succeeding plurality of virtual simulations and a real-time adaptation of the one or more graphical simulation artifacts based on the re-execution of the succeeding plurality of virtual simulations.

In some embodiments, the operations to calculate the weighted average include: setting a respective weight for each candidate trial of the plurality of candidate clinical trials based at least in part on a population size associated with the candidate trial or a similarity of one or more variables associated with the candidate trial to the plurality of distinct variable values extracted from the data associated with the configuration of the target clinical trial, wherein the operations to compute the composite statistical model include applying each respective weight against a respective statistical model for the candidate trial to generate a weighted statistical model and combining each weighted statistical model to generate the composite statistical model.

In some embodiments, the one or more variables each include a characteristic of a population, a characteristic of a drug, a design characteristic of a clinical trial, or a characteristic of a disease.

### BRIEF DESCRIPTION OF THE FIGURES

FIG. 1 illustrates a schematic representation of a system 100 in accordance with one or more embodiments of the present application;

FIG. 2 illustrates an example method 200 in accordance with one or more embodiments of the present application;

FIG. 3 illustrates an example preprocessing flow 300 in accordance with one or more embodiments of the present application;

FIG. 4 illustrates an example post-processing flow 400 in accordance with one or more embodiments of the present application;

FIG. 5 illustrates an example clinical trial relationship diagram 500 in accordance with embodiments of the present application; and

FIGS. 6-10 illustrate graphical user interface (GUI) views 600-1000 in accordance with embodiments of the present application.

### DESCRIPTION OF THE PREFERRED EMBODIMENTS

The following description of the preferred embodiments of the inventions are not intended to limit the inventions to these preferred embodiments, but rather to enable any person skilled in the art to make and use these inventions.

### 1.0 Clinical Trial Intelligence System

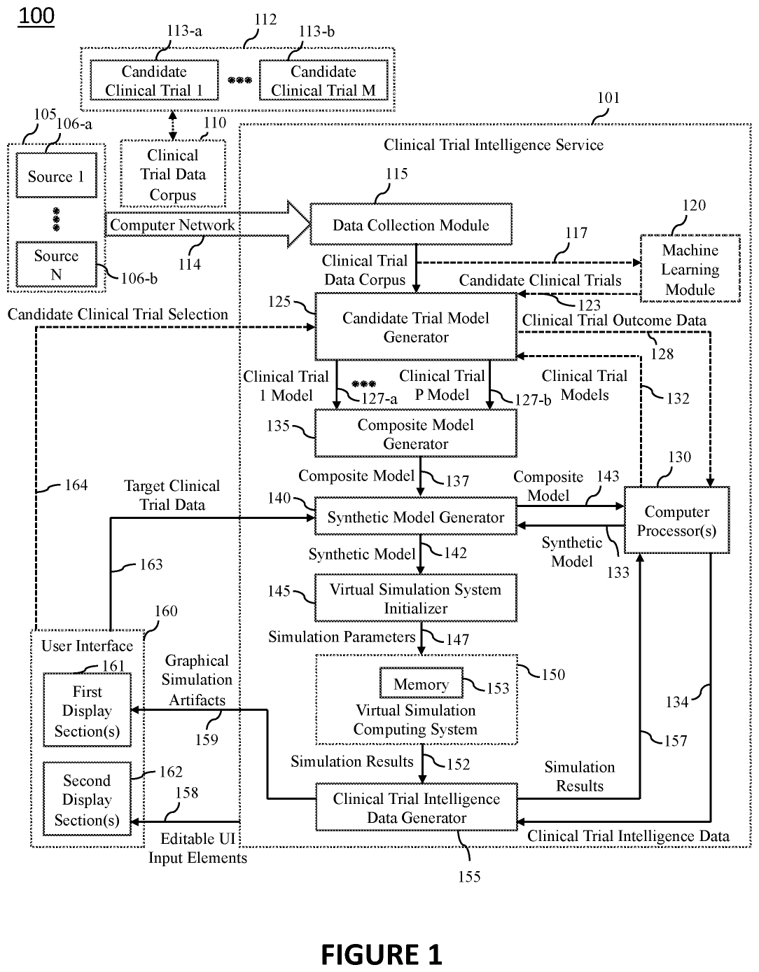

As shown in FIG. 1, a system 100 may include a remote clinical trial intelligence service 101, a source set 105 and a user interface 160. Clinical trial intelligence service 101 may include one or more of a data collection module 115, a machine learning module 120, a candidate trial model generator 125, computer processor(s) 130, composite model generator 135, synthetic model generator 140, virtual simulation system initializer 145, virtual simulation computing system 150, and clinical trial intelligence data generator 155. The clinical trial intelligence service 101 may be executed over a distributed network of computers (e.g., a cloud-based system) that is remotely accessible by subscribers and/or users of the clinical trial intelligence service 101 over a computer network that places one or more of a client application and/or client computer of a subscriber or user to the one or more of the network of computers implementing the clinical trial intelligence service 101.

Additionally, or alternatively, the modules of the clinical trial intelligence service 101 may be located on a single controller or may be located on any combination of multiple controllers configured to communicate with each other. In some examples, the user interface 160 may be located on a display device separate from but in communication with the clinical trial intelligence service 101. Additionally, or alternatively, the source set 105 may be retrieved from one or more memories or electronic data storage mediums of a system external to clinical trial intelligence service 101.

In some examples, clinical trial intelligence service 101 may be a cloud-based application that is hosted on one or more remote servers accessible via the user interface 160. In such examples, the user interface 160 may interact with the clinical trial intelligence service 101 using one or more network protocols (e.g., a Wireless Fidelity (Wi-Fi) protocol). The clinical trial intelligence service 101 may provide the user interface 160 to a user upon establishment of an initial connection with the clinical trial intelligence service 101 using the one or more network protocols. Accordingly, is shall be recognized that the modules of the clinical trial service 101 may be executed by the one or more network of computers or servers, which may specifically programmed or encoded to perform the several operations for generating clinical trial intelligence, controlling one or more simulations and/or predictive systems of the clinical trial service 101, and/or the like.

### 1.15 Data Collection Module

The data collection module 115 may function to collect (e.g., receive, obtain, retrieve) a clinical trial data corpus 110 from the source set 105. The clinical trial data corpus 110 may be collected from multiple sources from within the source set 105 (e.g., N sources, where N is a number greater than one). For instance, the clinical trial data corpus 110 may be collected from first source 106-a (e.g., Source 1) and a second source 106-b (e.g., Source N). In other examples, without deviating from the scope of the disclosure, the clinical trial data corpus may be collected from just one source (e.g., N is equal to one). The sources within the set 110 may be structured (e.g., a source with a standardized format, such as a database) or unstructured (e.g., a source with a non-standardized format). Examples of sources may include external databases, publications, interviews, and documents describing past results. Sources may be added to the source set 105 automatically or manually (e.g., through a user via a user interface). The clinical trial data corpus 110 may be obtained over a computer network 114.

The clinical trial data corpus 110 may be associated with a candidate clinical trial set 112, where the candidate clinical trial set 112 may include one or more candidate clinical trials (M clinical trials, where M is a number greater than or equal to 1). For instance, candidate clinical trial set 112 may include first candidate clinical trial 113-a (e.g., Candidate Clinical Trial 1) and second candidate clinical trial 113-b and second candidate clinical trial 113-b (e.g., Candidate Clinical Trial M).

Once collected by data collection module 115, the clinical trial data corpus may be stored in a repository (e.g., a document, a database) where it may be utilized by other modules of clinical trial intelligence service 101 (e.g., machine learning module 120, candidate trial model generator 125). Data collection module 115 may operate in real-time, ensuring that the repository is updated as additional sources are provided to the system.

### 1.20 Machine Learning Module

The machine learning module 120 may function to identify a subset of candidate clinical trials from the candidate clinical trial set 112 associated with the clinical trial data corpus 110. For instance, data collection module 115 may provide an indication 117 of the clinical trial data corpus 110 to the machine learning module 120, which in turn may provide the clinical trial data corpus 110 to a machine learning model of machine learning module 120. The machine learning model may be specifically trained to infer confidence values for clinical trials corresponding to a level of similarity between the clinical trials and a target clinical trial. The machine learning model may output the confidence values corresponding to the clinical trial data corpus 110 and may apply the confidence values to a minimum confidence value threshold to identify the subset of candidate clinical trials. Machine learning module 120 may provide an indication 123 of the subset of candidate clinical trials to candidate trial model generator 125.

It shall also be noted that, in some embodiments, to identify candidate clinical trials and/or to produce clinical trial intelligence data, the machine learning module 120 may function to implement any suitable machine learning model or any suitable ensemble of machine learning models. The one or more ensembles of machine learning models implemented by the system 100 may employ any suitable type of machine learning including one or more of: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), adversarial learning, and any other suitable learning style. Each module of the plurality can implement one or more of: a machine learning classifier, computer vision model, convolutional neural network (e.g., ResNet), visual transformer model (e.g., ViT), object detection model (e.g., R-CNN, YOLO, etc.), regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a semantic image segmentation model, an image instance segmentation model, a panoptic segmentation model, a keypoint detection model, a person segmentation model, an image captioning model, a 3D reconstruction model, a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, density-based spatial clustering of applications with noise (DBSCAN), expectation maximization, etc.), a bidirectional encoder representation from transformers (BERT) for masked language model tasks and next sentence prediction tasks and the like, variations of BERT (i.e., ULMFIT, XLM UDify, MT-DNN, SpanBERT, ROBERTa, XLNet, ERNIE, KnowBERT, VideoBERT, ERNIE BERT-wwm, MobileBERT, TinyBERT, GPT, GPT-2, GPT-3, GPT-4 (and all subsequent iterations), ELMo, content2Vec, and the like), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of machine learning algorithm. Each processing portion of the system 100 can additionally or alternatively leverage: a probabilistic module, heuristic module, deterministic module, or any other suitable module leveraging any other suitable computation method, machine learning method or combination thereof. However, any suitable machine learning approach can otherwise be incorporated in the system 100. Further, any suitable model (e.g., machine learning, non-machine learning, etc.) may be implemented in the various systems and/or methods described herein.

### 1.25 Candidate Trial Model Generator

The candidate trial model generator 125 may function to derive (e.g., generate, compute) a statistical model for each of a set of candidate clinical trials (e.g., a set of P clinical trials, where P is an integer greater than or equal to 1). For instance, the candidate trial model generator 125 may derive a first clinical trial statistical model 127-a (e.g., Clinical Trial 1 Model) and a second clinical trial statistical model 127-b (e.g., Clinical Trial P Model).

In some examples, the candidate trial model generator 125 may receive an indication 117 of the clinical trial data corpus 110 from the data collection module 115 and may determine the set of candidate clinical trials from the clinical trial data corpus 110 (e.g., may determine the set of candidate clinical trials as candidate clinical trial set 112, where P=M). In other examples, the candidate trial model generator 125 may receive the indication 123 of the subset of candidate clinical trials from machine learning module 120 and may determine the set of candidate clinical trials as the indicated subset of candidate clinical trials (e.g., P is less than or equal to M).

In either case, deriving the statistical model for each of the set of candidate clinical trials may include extracting, from the clinical trial data corpus 110, outcome data for a set of samples in the candidate clinical trial, where the outcome data includes an outcome for each sample in the set of samples. Additionally, the deriving may include executing, by one or more computer processors, a regression on the set of samples to generate a probability distribution for the candidate clinical trial corresponding to the outcome data. For instance, candidate trial model generator 125 may provide, to computer processor(s) 130, an indication 128 of clinical trial outcome data for each of the set of candidate clinical trials. Computer processor(s) 130 may execute the regression on the set of samples for the clinical trial outcome data associated with each candidate clinical trial and may return an indication 132 of a set of clinical trial statistical models.

### 1.30 Computer Processor(s)

The computer processor(s) 130 may function to retrieve, process, and manage data or instructions from other modules of the clinical trial intelligence system 101. The computer processor(s) 130 may include a single processing unit, or, alternatively, may encompass multiple processing units that function in parallel or independently. In embodiments where multiple processors are utilized, these processors may reside on the same physical host system or may be distributed across multiple host systems, potentially located in geographically disparate locations. This configuration allows for enhanced processing flexibility and scalability, as the distribution of processing tasks can be optimized based on available system resources and computational demands.

In certain implementations, the computer processor(s) 130 may be specifically configured to allocate specific subsets of processing units to particular functions or modules thereby forming one or more distinct microservices, which may be specifically configured with software applications, scripts, computer logic, and/or control instructions for executing the plurality of modules within the clinical trial intelligence system 101. For example, a dedicated processor or subset of processors may be specifically assigned and/or programmed to perform operations associated with data collection module 115, while another processor or subset of processors may be tasked with operations associated with candidate trial model generator 125. This modular allocation of processing resources can facilitate efficient parallel processing, reduce latency, and improve overall system throughput by ensuring that specialized processors handle designated tasks.

Additionally, the computer processor(s) 130 may be configured with various control logic and processing pipelines that optimize data flow between system components. In embodiments involving multi-core processors or multi-processor systems, individual cores or processors may be dynamically assigned to handle computationally intensive tasks. These processors may further support various modes of operation, such as single-instruction-multiple-data (SIMD) or multi-threading, enabling simultaneous processing of large datasets and further enhancing system performance.

In some instances, the computer processor(s) 130 may include specialized processors, such as graphics processing units (GPUs), tensor processing units (TPUs), or other application-specific integrated circuits (ASICs), to perform specialized functions, such as machine learning model training, large-scale data analytics, or secure computation. The inclusion of such specialized processors can further optimize the performance of the clinical trial intelligence system 101 in specific application domains, ensuring that tasks requiring high computational power are executed efficiently.

### 1.35 Composite Model Generator

The composite model generator 135 may function to compute a composite statistical model 137 based on calculating a weighted average of a combination of the statistical model for each of the set of candidate clinical trials for which a statistical model was generated. For instance, composite model generator 135 may receive an indication of a first statistical model 127-a and a second statistical model 127-b as part of a set of P statistical models. Composite model generator 135 may compute a composite statistical model 137 from the set of P statistical models by calculating a weighted average of a combination of the set of P statistical models.

In some examples, calculating the weighted average includes composite model generator 135 setting a respective weight for each candidate trial of the set of candidate clinical trials (e.g., a first weight for first statistical model 127-a and a second weight for second statistical model 127-b). The weights may be assigned to a respective candidate trial based on a population size associated with the candidate trial (e.g., larger weights for candidate trials with a higher population size), a similarity of one or more first variable values associated with the candidate trial to a set of one or more second variable values associated with a configuration of a target clinical trial (e.g., more similarity between the target clinical trial and the candidate clinical trial may result in a larger weight), or both. The one or more variables with the first and second values may include a population size, a characteristic of a drug, a design characteristic of a clinical trial, or a characteristic of a disease.

Additionally, calculating the weighted average may include applying each respective weight against a respective statistical model for a candidate trial (e.g., multiplying a probability distribution of a statistical model by a corresponding weight) to generate a weighted statistical model and combining (e.g., averaging) each weighted statistical model to generate the composite statistical model. For instance, a first computed weight may be applied against first statistical model 127-a to generate a first weighted statistical model and a second computed weight may be applied against second statistical model 127-b to generate a second statistical model 127-b. The first weighted statistical model and the second weighted statistical model may be averaged to generate a corresponding composite statistical model 137.

### 1.40 Synthetic Model Generator

The synthetic model generator 140 may function to generate a synthetic statistical model 142 for a target clinical trial based on adapting the composite statistical model 137 using a set of distinct variable values extracted from data associated with a configuration of the target clinical trial. For instance, synthetic model generator 140 may receive an indication of a composite model 137 generated by composite model generator 125 and may obtain target clinical trial data. The synthetic model generator 140 may provide an indication 143 of the composite model 137 to computer processor(s) 130, which may generate the synthetic statistical model 142 using the provided indication 143. The computer processor(s) 130 may provide an indication 133 of the synthetic statistical model 142 to synthetic model generator 140. The target clinical trial data may be obtained as target clinical trial data 163 via user interface 160 (e.g., for an initial configuration or a reconfiguration of the target clinical trial via user interface 160) or may be retrieved from memory in communication with or included as part of clinical trial intelligence service 101 (e.g., for an initial configuration of the target clinical trial). The set of distinct variables. Values may each include a population size, a characteristic of a drug, a design characteristic of a clinical trial, or a characteristic of a disease.

In some examples, generating the synthetic statistical model 142 may include extracting the set of distinct variables from the data associated with the configuration of the target clinical trial data (e.g., from target clinical trial data 163) and transforming the composite statistical model 136 into the synthetic statistical model 142 based on applying the set of distinct variable values to the composite statistical model 137. For instance, the distinct variable values may be used to adjust parameters or descriptors defining the composite statistical model 137 such that the adjusted parameters define the synthetic statistical model 142.

### 1.45 Virtual Simulation System Initializer

The virtual simulation system initializer 145 may function to initialize a virtual simulation computing system (e.g., virtual simulation computing system 150) with a set of simulation parameters 147. The set of simulation parameters 147 may include a representation of the synthetic statistical model 142 (e.g., a probability distribution) and a set of input values for a set of variables associated with the synthetic statistical model 142.

In some examples, initializing the virtual simulation computing system 150 with the set of simulation parameters 147 may include loading the representation of the synthetic statistical model 142 and the set of input values for the set of variables associated with the synthetic statistical model 142 into a memory 153 accessible to one or more specifically configured and/or programmed computer processors of virtual simulation computing system 150 that, based on the initialization with the set of simulation parameters 147, automatically construct a plurality of virtual clinical trial simulations (e.g., thousands, tens of thousands, and/or more). The representation of the synthetic statistical model 142 and the set of input values for the set of variables associated with the synthetic statistical model 142 may be retrievable from memory 153 for executing the set of virtual simulations.

In other examples, the set of simulation parameters 147 include the set of candidate trials for which a statistical model is generated (e.g., which may be configurable via an editable UI elements 158 via selection of the set of candidate trials as described herein). In such examples, initializing the virtual simulation computing system 150 may include loading the set of candidate trials into a memory of clinical trial intelligence service 101 (e.g., memory 153), where the set of candidates may be retrievable from the memory for computing or re-computing the composite statistical model 137.

### 1.50 Virtual Simulation Computing System

The virtual simulation computing system 150, as specifically configured, may function to execute a set of virtual simulations based on the initialization of the virtual simulation computing system 150 with the set of simulation parameters 147 (e.g., via virtual simulation system initializer 145). In some examples, virtual simulation computing system 150 may be a computing system specifically configured and/or programmed with unique simulation computer instructions designed to optimize a generation of a plurality simulations, such that the plurality of simulations (e.g., thousands of virtual simulations) may be performed in real-time, efficiently and with high accuracy exceeding standard accuracy of typical simulation or data processing systems. For instance, virtual simulation computing system 150 may include a set of processors and/or a network of computers optimized or otherwise configured for performing simulation-related tasks in parallel. Additionally, the virtual simulation computing system 150 may include memory, such as memory 153, dedicated to storing information for simulation-related tasks (e.g., memory that stores the set of simulation parameters 147).

### 1.55 Clinical Trial Intelligence Data Generator

The clinical trial intelligence data generator 155 may function to generate clinical trial intelligence data 155 based on the execution of the set of virtual simulations. For instance, clinical trial intelligence data generator may receive from virtual simulation computing system 150, simulation results 152 and may provide an indication 157 of the simulation results 152 to processor(s) 130. The processor(s) 130 may generate clinical trial intelligence data from the simulation results 152 and may provide an indication 134 of the clinical trial intelligence data to clinical trial intelligence data generator 155. The clinical intelligence data may include graphical simulation artifacts 159.

### 1.60 User Interface

The user interface (UI) 160 may be a graphical user interface (GUI) and may function to control and/or configure the clinical trial intelligence service 101. In some examples, the UI 160 may display one or more graphical simulation artifacts in the one or more first display sections 161. Additionally, the UI 160 may display editable user interface input elements. The one or more graphical simulation artifacts 159 and the editable UI input elements may be provided by the clinical trial intelligence service 101.

The editable UI input elements 158, when manipulated, may configure or reconfigure one or more of the simulation parameters 147. The configuration or reconfiguration of the one or more of the simulation parameters 147 may enable a re-execution of a succeeding set of virtual simulations and a real-time adaptation of the one or more graphical simulation artifacts based on the re-execution of the succeeding set of virtual simulations. For instance, when one or more simulation parameters 147 are configured (or re-configured) the virtual simulation system initializer 145 may initialize the virtual simulation computing system 150 with the updated set of simulation parameters 147. The virtual simulation computing system 150 may execute a succeeding set of virtual simulations corresponding to the updated set of simulation parameters 147 and the updated simulation results may be provided to clinical trial intelligence data generator 155. Clinical trial intelligence data generator 155 may generate, in real-time, an updated clinical trial intelligence data including updates to the one or more graphical simulation artifacts.

In some examples, the editable UI input elements 158 may configure or reconfigure the one or more simulation parameters 147 by sending one or more input or control signals to one or more computer processors of the clinical trial intelligence service 101 that trigger or automatically causes the clinical trial intelligence service 101 to add a candidate clinical trial to the set of candidate clinical trials for which statistical models may be generated or to remove a candidate clinical trial from the set of candidate clinical trials thereby automatically reconfiguring the synthetic statistical model of a target clinical trial. Upon adding or removing the candidate clinical trial, re-computation of the composite statistical model may occur for re-execution of the succeeding set of virtual simulations. In order to facilitate the adding or removing of candidate clinical trials, the editable UI input elements 158 may be configured such that a user may select which candidate clinical trials should be added to or removed from the candidate clinical trials for which statistical models are generated by candidate trial model generator 125. For instance, each of a subset of the editable UI input element 158 may correspond to a respective candidate clinical trial and may be toggled by a user. Additionally, if a candidate clinical trial is associated with multiple trial arms (e.g., an intervention trial arm and a control trial arm), the UI input element 158 may be configured such that a user may select one trial arm or both. After performing the toggling, the UI 160 may provide an indication of a candidate clinical trial selection 164 to candidate trial model generator 125. Candidate trial model generator 125 may re-generate statistical models for the selected candidate clinical trials and may provide the statistical models to composite model generator 137. Composite model generator 137 may generate an updated composite statistical model and may provide the updated composite statistical model to synthetic model generator 140. Synthetic model generator 140 may use the updated composite statistical model to generate an updated synthetic statistical model, which in turn may indicate an updated set of simulation parameters.

In some examples, the set of editable UI input elements 158 may configure or reconfigure the one or more simulation parameters 147 by triggering the clinical trial intelligence service 101 to update at least one of the set of distinct variable values associated with the configuration of the target clinical trial. Upon updating at least one of the set of distinct variable values associated with the configuration, re-generating of the synthetic statistical model 142 may occur for re-execution of the succeeding set of virtual simulations. In order to facilitate the updating of at least one of the set of distinct variable values, the editable UI input elements 158 may be configured such that a user may select which distinct variable value should be updated. For instance, each of a subset of the editable UI input element 158 may correspond to a respective variable value that may be adjusted by a user. After performing the adjusting, the UI 160 may provide an indication of updated target clinical trial data 163 (e.g., updated distinct variable values) to clinical trial intelligence service 101 (e.g., to synthetic model generator 140). Synthetic model generator 140 may use the updated distinct variable values to generate an updated synthetic statistical model.

In some examples, the graphical simulation artifacts may include information corresponding to multiple clinical trial arms. In order to facilitate this inclusion, the data collection module 115 may obtain, via the computer network from one or more second sources of digital data, a second corpus of historical trial data associated with a second set of candidate clinical trials. It should be noted that, in some examples, there may be overlap between the one or more second sources of digital data and the sources of source set 105. Additionally, it should be noted that, in some examples, there may be overlap between the candidate clinical trials of candidate clinical trial set 112 and the second set of candidate clinical trials.

The candidate trial model generator 125 may derive a statistical model for each of the second set of candidate clinical trials based on the second corpus of historical clinical trial data associated with each of the second set of candidate clinical trials. The composite model generator 135 may compute a second composite statistical model based on calculating a weighted average of a combination of the statistical model for each of the second set of candidate clinical trials. The synthetic model generator 140 may generate (e.g., via computer processor(s) 130) a second synthetic statistical model for the target clinical trial based on adapting the second composite statistical model using a second set of distinct variable values extracted from the data associated with the configuration of the target clinical trial. The virtual simulation system initializer 145 may initialize the virtual simulation computing system 150 with a second set of simulation parameters including a representation of the second synthetic statistical model and a set of input values for a set of variables associated with the second synthetic statistical model. The virtual simulation computing system 150 may execute a second set of virtual simulations based on the initialization of the virtual simulation computing system 150 and may provide second simulation results to clinical trial intelligence data generator 155. Clinical trial intelligence data generator may include data corresponding to both the set of simulation parameters 147 and the second set of simulation parameters, where the set of simulation parameters 147 may correspond to a first clinical trial arm (e.g., an intervention trial arm) of the target clinical trial and the second set of simulation parameters may correspond to a second clinical trial arm (e.g., a control trial arm). Without deviating from the scope of the disclosure, it should be noted that there may be examples of the techniques described herein in which only a single-arm is simulated (e.g., an intervention-only simulation), such as via a single set of simulation parameters. Additionally, it should be noted that there may be examples in which more than two arms are simulated (e.g., a control trial arm and multiple intervention trial arms), such as via three or more sets of simulation parameters.

### 2.00 Method for Implementing Interactive Graphical User Interfaces for Accelerated Integration and Manipulation of Clinical Trial Data

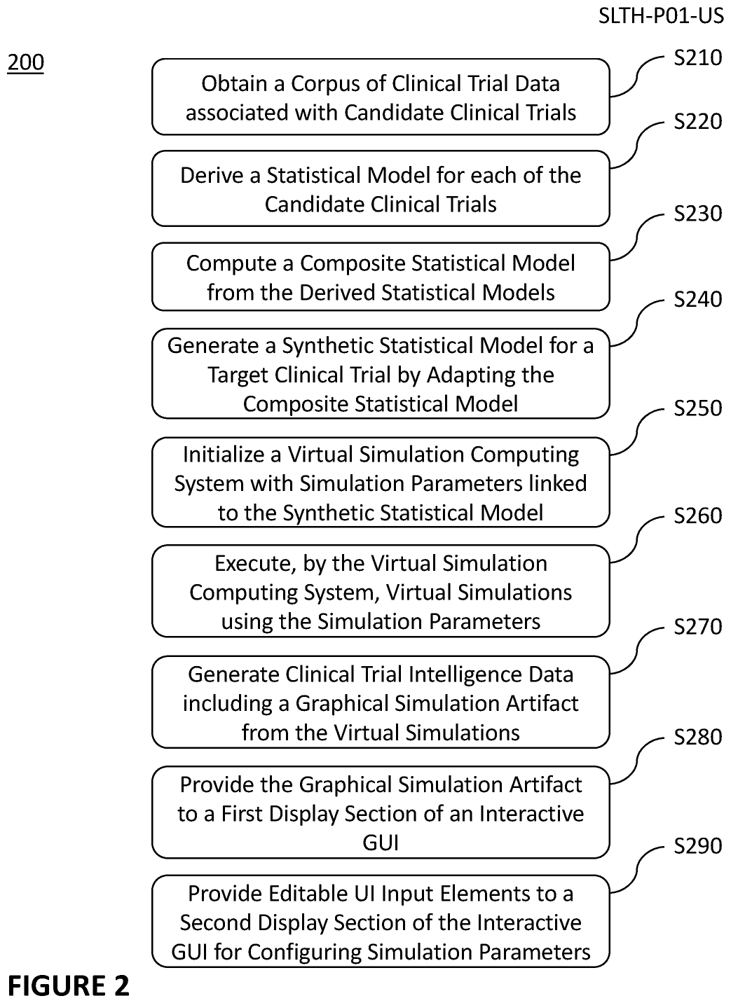

As shown in FIG. 2, method 200 for implementing GUIs for accelerated integration and manipulation of clinical trial data may include deriving a statistical model for each of the candidate clinical trials; computing a composite statistical model from the derived statistical models; generating a synthetic statistical model for a target clinical trial by adapting the composite statistical model; initializing a virtual simulation computing system with simulation parameters linked to the synthetic statistical model; executing, by the virtual simulation computing system, virtual simulations using the simulation parameters; generating clinical trial intelligence data including a graphical simulation artifact from the virtual simulations; providing the graphical simulation artifact to a first display section of an interactive GUI; and providing editable UI input elements to a second display section of the interactive GUI for configuring simulation parameters. It shall be appreciated that other examples contemplated within the scope of the present disclosure may involve more operations, fewer operations, different operations, or a different order of operations than as shown in FIG. 2.

The techniques described herein enhance user interaction through an interactive and dynamic graphical user interface (GUI) that allows users to configure and reconfigure simulation parameters in real time. This interactive system enables users to manipulate values of the simulation parameters via UI input elements and immediately see the impact of those manipulations on the simulated trial outcomes. This real-time adaptability makes clinical trial simulations more flexible, responsive, and user-friendly as compared to static, non-interactive systems.

### 2.10 Obtaining a Corpus of Clinical Trial Data

S210, which includes obtaining, via a computer network from one or more sources of digital data, a corpus of historical clinical trial data associated with each of a set of candidate clinical trials, may function to aggregate data from clinical trials. The one or more sources of digital data may be in a structured format or an unstructured format. Such sources may include databases, publications, interviews, and documentation describing past results of clinical trials. Additionally, previous results of previously performed simulations may serve as a source. S210 may be performed, in some examples, by a data collection module 115 as described with reference to FIG. 1.

The term “clinical trial” may be defined as a research study conducted over a population of patients to evaluate the safety, efficacy, and potential benefits of medical interventions. The term “clinical trial data” may refer to information about a particular clinical trial, including results and outcomes linked to the clinical trial. The term “historical” in historical clinical trial data may refer to the clinical trial data being associated with clinical trials whose outcomes have already occurred (e.g., clinical trials that have been concluded). The term “corpus” refers to information retrieved from a set of sources that has been collected or aggregated into a structured format. A “candidate clinical trial” may be defined as a clinical trial whose information (e.g., results and outcomes) is being considered for simulation of a target clinical trial.

A computer network as defined herein may refer to a system of interconnected devices, such as computers, servers, and other hardware, that communicate and share resources (e.g., data, applications, or hardware) with each other. These devices are linked through communication channels, such as wired or wireless connections, enabling data exchange and resource access across the network. Networks can vary in size and scope, ranging from small local area networks (LANs) that connect devices within a limited area, to wide area networks (WANs) that cover broader geographical regions, including the internet.

### 2.20 Deriving Statistical Models for Candidate Clinical Trials

S220, which includes deriving a statistical model for each of the set of candidate clinical trials based on the corpus of historical clinical trial data associated with each of the set of candidate clinical trials, may function to convert the historical clinical trial data into a set of statistical models. In some examples, deriving the statistical model may include extracting, from the corpus of historical clinical trial data, outcome data for a set of samples in the candidate clinical trial and executing, by one or more computer processors, a regression on the set of samples to generate a probability distribution for the candidate clinical trial corresponding to the outcome data. S220 may be performed, in some examples, by a candidate model trial generator 125 as described with reference to FIG. 1.

A statistical model may be defined as a mathematical framework that represents data and relationships between variables. A statistical model for a candidate clinical trial may represent outcome data for a clinical trial as a relationship between a patient population and an outcome metric. Outcome data may, for instance, include values of metrics corresponding to outcomes for a patient population participating in the clinical trial, such as progression-free or overall survival over a period of time. A value of the metric for a particular patient of the patient population may be referred to as a sample, where a set of samples may represent the value of the metric for some or all of the patient population. Regression may be a technique that may be performed on the set of samples in order to construct a model that may be used to generate a probability distribution corresponding to the set of samples.

Preprocessing flow 300 may illustrate a non-limiting example of a flow for transforming a corpus of historical clinical trial data into statistical models. For instance, a set of sources (e.g., information sources) may be provided for preprocessing 310. The set of sources in the present example may include databases 305-a, publications 305-b, interviews 305-c, and past results 305-d (e.g., past clinical trial outcomes, past clinical trial arm information). Performing the preprocessing 310 may result in base models 315, model variables 320, and similar trial identification 325 (e.g., determining a similarity ranking for clinical trial arms).

In some examples, preprocessing flow 300 may be performed by one or more aspects of FIG. 1. For instance, a data collection module 115 may collect and/or aggregate the set of sources and candidate trial model generator 125 may transform information collected from the set of sources into a set of base models, a set of model variables, a ranking of clinical trial arms, or a combination thereof.

Each base model 315 may serve as a statistical framework for understanding likely outcomes of a clinical trial and may represent a known outcome of a candidate clinical trial. Constructing a base model 315 may include transforming information extracted from the set of sources into a probability distribution. Each model variable 320 may represent specific factors that may influence the outcome of a clinical trial and may be derived from trial-specific data (e.g., patient demographics, biomarkers, treatment regimens, disease severity). Each variable may be associated with a range of potential values that may modify a base model 315 and tailor them to a clinical trial of interest. These variables may introduce flexibility into a predictive modeling process, allowing the base models 315 to be adjusted for different clinical trial scenarios. Constructing a model variable may include transforming information extracted from one or more sources of the set of sources into a range of values and an indication of a corresponding influence (e.g., impact) that such a variable may have on a base model 315.

In order to perform similarity trial identification 325, a similarity algorithm that compares clinical trials according to a set of factors may be performed and a similarity score for each clinical trial may be assigned for each clinical trial based on how closely its characteristics match other clinical trials (e.g., the target clinical trial). The similarity score may be calculated using a deterministic technique or via a machine learning model. Once the similarity scores are calculated, the clinical trials may be ranked in order of relevance to the target clinical trial. An indication of the similarity scores and/or the ranking may be displayed on a GUI (e.g., a GUI displaying clinical trial intelligence data).

In some examples, dynamic ranking of candidate clinical trials may be utilized. For instance, the ranking may be adjusted over time as new candidate clinical trials are added or as candidate clinical trials are removed. Additionally, feedback (e.g., received via a GUI) may be used to adjust aspects of the algorithm that determines the similarity scores. For instance, the feedback may indicate a quality and relevance of candidate clinical trials ranked as most similar to a target clinical trial and may be used to adjust weights applied to one or more factors in future similarity analyses, thus ensuring that identification and ranking of similar trials improves over time.

In some examples, the calculated similarity scores may be used for automatic selection of a subset of candidate clinical trials (e.g., via machine learning module 120 of FIG. 1). For instance, clinical trials with a higher similarity score to a target clinical trial may be assigned a higher weight when generating a composite statistical model. Additionally, feedback may be received

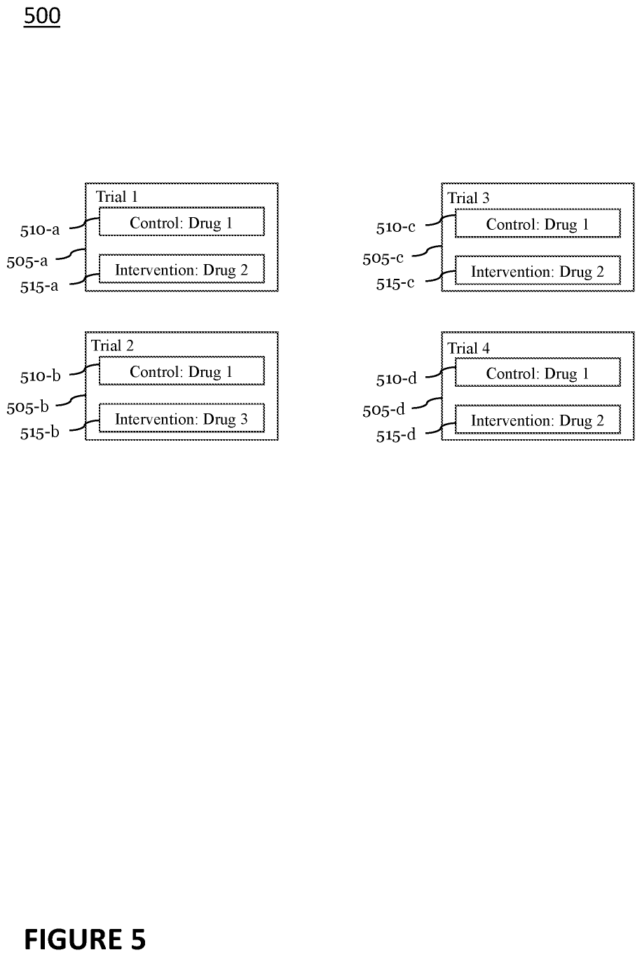

Clinical trial relationship diagram 500 of FIG. 5 may illustrate a non-limiting example of similar trial identification (e.g., as corresponding to similar trial identification 325). For instance, clinical trial relationship diagram 500 may depict clinical trials 505-a, 505-b, 505-c, and 505-d. Clinical trial 505-a may have a control arm 510-a treated with drug 1 and an intervention arm 515-a treated with drug 2. Clinical trial 505-b may have a control arm 510-b treated with drug 1 and an intervention arm 515-b treated with drug 2. Clinical trial 505-c may have a control arm 510-c treated with drug 3 and an intervention arm 515-c treated with drug 4. Clinical trial 505-d may have a control arm 510-d treated with drug 1 and an intervention arm 515-d treated with drugs 2 and 3.

Each clinical trial arm may represent a group of patients in a clinical trial. The control clinical trial arm (e.g., control arms 510-a through 510-d) may serve as the baseline or standard arm and may, for instance, correspond to a placebo treatment or an active treatment whose effects are already well understood. The intervention clinical trial arm (e.g., intervention arms 515-a through 515-d), meanwhile, may serve as a new or less well understood treatment as compared to the control clinical trial arm.

Due to intervention arm 515-b being treated with drug 3, information, intervention arm 515-b may be more likely to be similar to control arm 510-c as opposed to other clinical trial arms that do not utilize drug 3. Thus, intervention arm 515-b may be ranked as being more similar to control arm 510-c and/or may be capable of having a larger influence in simulating control arm 510-c if clinical trial 505-c is a virtual trial. If intervention arm 515-b is similar to intervention arm 515-c (e.g., drug 3 is similar to drug 4, both arms have similar factors or conditions), intervention arm 515-b may likewise be ranked as being more similar to intervention arm 515-c and/or may be capable of having a larger influence in simulating intervention arm 515-c if clinical trial 505-c is a virtual trial.

Due to control arms 510-a and 510-b being treated with drug 1, control arms 510-a and 510-b may be more likely to be similar to control arm 510-d as compared to other clinical trials that do not utilize drug 1. Thus, control arms 510-a and 510-b may be ranked as being more similar to control arm 510-d and/or may be capable of having a larger influence in simulating control arm 510-d if clinical trial 505-d is a virtual trial. Additionally, due to intervention arm 515-a being treated with drug 2 and intervention arm 515-b being treated with drug 3, intervention arms 515-a and 515-b may be more likely to be similar to intervention arm 515-d as compared to other clinical trials that do not utilize drugs 2 or 3. Thus, intervention arms 515-a and 515-b may be ranked as being more similar to intervention arm 515-d and/or may be capable of having a larger influence in simulating intervention arm 515-d if clinical trial 505-d is a virtual trial.

### 2.30 Computing a Composite Statistical Model

S230, which includes computing a composite statistical model based on calculating a weighted average of a combination of the statistical model for each of the set of candidate clinical trials, may function to generate a composite statistical model representing a weighted contribution of each of the set of candidate clinical trials. In some examples, calculating the weighted average include setting a respective weight for each candidate trial of the set of candidate trials based on a population size associated with the candidate trial or a similarity of one or more variables associated with the candidate trial to the set of distinct variable values extracted from the data associated with the configuration of a target clinical trial. In such cases, computing the composite statistical model includes applying each respective weight against a respective statistical model for the candidate trial to generate a weighted statistical model and averaging each weighted statistical model to generate the composite statistical model. S230 may be performed, in some examples, by a composite model generator 135 as described with reference to FIG. 1.

The term “composite statistical model” may refer to a statistical model that combines two or more individual statistical models into a single framework. The term “population size” may refer to the size of a patient population associated with a clinical trial. Setting the respective weight for a candidate clinical trial based on population size may involve utilizing a deterministic function or a machine learning model that assigns a higher weight for a higher population size and a lower weight for a lower population. Similarity between the one or more variable values may refer to a measure of how close the one or more variable values are (e.g., a difference between the variable values).

The one or more variables may be measures of a population (e.g., measures of a patient population), a characteristic of a drug, a design characteristic of a clinical trial, or a characteristic of a disease. Measures of a population may include, for instance, a population size, age, or a percentage of the population that fits within a particular category (e.g., a percentage of the population that smokes, has metastatic growth). Measures of a characteristic of a drug may, for instance, include dosage and concentration. Measures of a design characteristic of a clinical trial may, for instance, include a duration of a clinical trial. Measures of a characteristic of a disease may include, for instance, a disease stage or the presence of biomarkers or inflammatory markers.

### 2.40 Generating a Synthetic Statistical Model for a Target Clinical Trial

S240, which includes generating, by one or more computer processors (e.g., processor(s) 130), a synthetic statistical model for a target clinical trial, may function to compute a statistical model that adapts the composite statistical model for the target clinical trial. For instance, generating the synthetic statistical model may be based on adapting the composite statistical model using a set of distinct variable values extracted from data associated with a configuration of the target clinical trial. Generating the synthetic statistical model may include extracting the set of distinct variable values from the data associated with the configuration of the target clinical trial data and transforming the composite statistical model into the synthetic statistical model based on applying the set of distinct variable values to the composite statistical model. S240 may be performed, in some examples, by a synthetic model generator 140 as described with reference to FIG. 1.

A synthetic statistical model as described herein may refer to a statistical model that predicts the statistical model that would be generated for a target clinical trial if the target clinical trial were, for instance, included in the set of candidate clinical trials. The term “target clinical trial” may refer to the clinical trial that is being simulated and may, in some examples, refer to a clinical trial whose outcome data the clinical trial intelligence service does not have access to (e.g., a clinical trial whose outcome has not yet been determined). The variables associated with the set of distinct variable values may, for instance, be measures of a population (e.g., measures of a patient population), a characteristic of a drug, a design characteristic of a clinical trial, or a characteristic of a disease as described herein.

In some examples, applying the set of distinct variable values to the composite statistical model may include re-estimating one or more parameters of the composite statistical model based on the set of distinct variable values. For instance, coefficients associated with the composite statistical model (e.g., regression model coefficients) may be adjusted via application of the set of distinct variable values. To the composite statistical model.

### 2.50 Initializing a Virtual Simulation Computing System

S250, which includes initializing a virtual computing system with a set of simulation parameters, may function to prepare the virtual computing system for performing a set of virtual simulations. The set of simulation parameters may include a representation of the synthetic statistical model and a set of input values for a set of variables associated with the synthetic statistical model. In some examples, initializing the virtual simulation computing system may include loading the representation of the synthetic statistical model and the set of input values for the set of variables associated with the synthetic statistical model into a memory of the virtual simulation computing system, where the representation of the synthetic statistical model and the set of input values may be retrievable form memory for executing a set of virtual simulations. S250 may be performed, in some examples, by a virtual simulation system initializer 145 as described with reference to FIG. 1.

A representation of a synthetic statistical model may, for instance, include a probability distribution. The set of variables may, for instance, represent one or input variables to the synthetic statistical model and the set of input values may correspond to the values of the one or more input variables. In some examples, the set of input values may be the same as the distinct variable values used to generate the synthetic statistical model.

### 2.60 Executing Virtual Simulations by the Virtual Simulation Computing System

S260, which includes executing, by the virtual simulation computing system, a set of virtual simulations based on the initialization of the virtual simulation computing system with the set of simulation parameters, may function to generate simulation results for the target clinical trial. S260 may be performed, in some examples, by a virtual simulation computing system 150 as described with reference to FIG. 1.

Performing a virtual simulation may include the virtual simulation computing system running a sequence of trials using the synthetic statistical model. Running each trial of the sequence of trials may include generating a set of samples for a population of patients using the synthetic statistical model, where each sample corresponds to an outcome for a patient of the population of patients. The results of each trial of the sequence of trials may be aggregated and processed to generate simulation results where the simulation results are utilized for the generation of clinical trial intelligence data. The simulation results may include measures of statistical significance, a confidence interval, a probability distribution over a range of possible outcomes, and other measures of effectiveness (e.g., hazard ratios).

Post-processing flow 400 may illustrate a non-limiting example of a flow for executing virtual simulations. For instance, base models 405, model variables 410, and similar trials identified in similar trial identification 415 may be run through simulation 420. The simulation may be based on a set of probability distributions derived from base models 405 and model variables 410. Raw results of the simulation 420 may undergo post-processing 425.

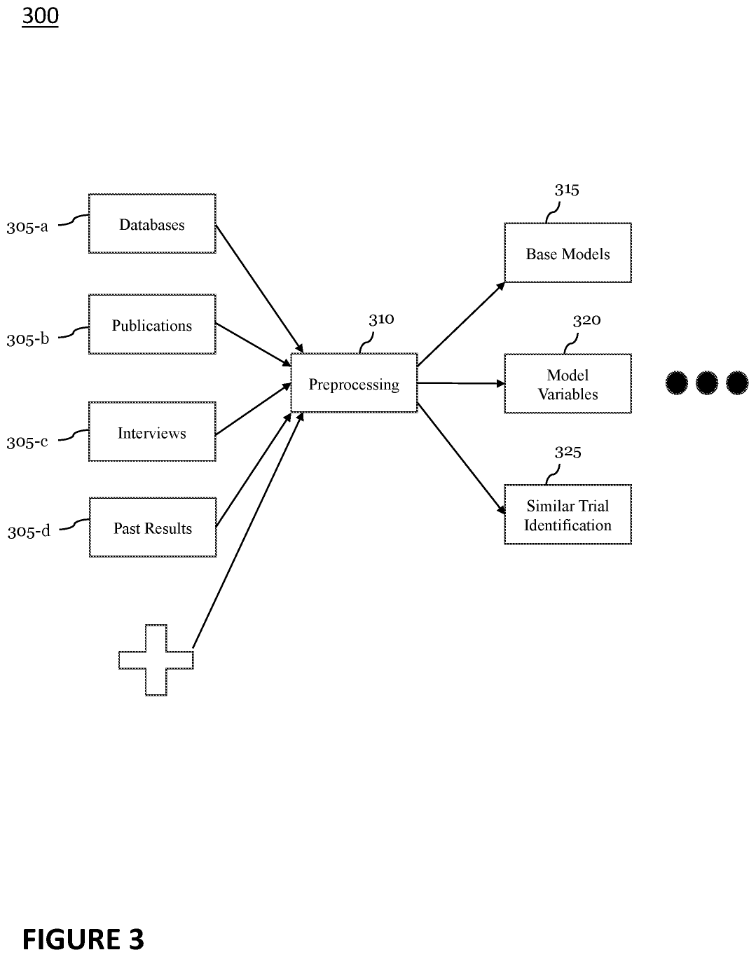

In some examples, simulation 420 may be performed by one or more aspects of FIG. 1. For instance, simulation 420 may be performed by virtual simulation computing system 150. Additionally, base models 405 may be an example of base models 315 as described with reference to FIG. 3, model variables 410 may be an example of model variables 320 as described with reference to FIG. 3, and similar trial identification 415 may be an example of similar trial identification 325 as described with reference to FIG. 3.

### 2.70 Generating Clinical Trial Intelligence Data

S270, which includes generating, by the one or more computer processors (e.g., computer processor(s) 130), clinical trial intelligence data based on the execution of the set of virtual simulations, may function to provide information that estimates the outcome of the target clinical trial. The clinical trial intelligence data may include one or more graphical simulation artifacts. S270 may be performed, in some examples, by a clinical trial intelligence data generator 155 as described with reference to FIG. 1.

Post-processing flow 400 may illustrate a non-limiting example of a flow for generating an output from a simulated clinical trial. For instance, the raw results received from simulation may undergo post-processing 430, which may generate model outputs to be provided to user interface 425. The model outputs may include a graphical simulation artifact as described herein. In some examples, post-processing 425 may be performed by one or more aspects of FIG. 1. For instance, clinical trial intelligence data generator 155 of FIG. 1 may perform the post-processing 425 as described herein. Additionally, user interface 425 may correspond to user interface 160 of FIG. 1.

### 2.80 Providing a Graphical Simulation Artifact to an Interactive GUI

S280, which includes providing, to at least a first display section of an interactive simulation GUI, the one or more graphical simulation artifacts, may function to display a graphical representation of the clinical trial intelligence data. S280 may be performed, in some examples, by a clinical trial intelligence service 101 as described with reference to FIG. 1.

One example of a graphical simulation artifact may include a survival curve (e.g., a Kaplan-Meier survival curve) of overall survival or progression-free survival, where a Kaplan-Meier survival curve may display a proportion of patients who remain alive or progression-free, respectively, over time. Another example of a graphical simulation artifact may be a cumulative distribution function showing probability distribution of survival times. Other graphical simulation artifacts that provide a visual summary of patient outcomes over time may, additionally or alternatively, be generated (e.g., a graphical simulation artifact corresponding to another clinical trial endpoint measurement).

In some examples, clinical trial intelligence data may include a numerical simulation artifact and S280 may include providing the numerical simulation artifact to at least the first display section. For instance, the numerical simulation artifact may include a table or another UI element that indicates a median survival time, which may represent a length of time from the start of treatment that half of the patients diagnosed with a particular condition are still alive or are still progression-free. Additionally, or alternatively, the numerical simulation artifact may include a table or another UI element that indicates a hazard ratio, which may measure a survival rate of the intervention clinical trial arm relative to the control clinical trial arm. Additionally, or alternatively, the numerical simulation artifact may include a table or another UI element that indicates a percent survival after a given quantity of months (e.g., 6-month survival, 12-month survival) and/or a p-value, the latter of which may be a measure quantifying a probability of obtaining a value as extreme as or more extreme than one that is observed.

In some examples, clinical trial intelligence data may include a categorical simulation artifact and S280 may include providing the categorical simulation artifact to at least the first display section. For instance, the categorical simulation artifact may include a UI element that indicates a success or failure of a target clinical trial. A success may be indicated if an intervention clinical trial arm shows a statistical improvement in survival time or progression-free survival relative to the control clinical trial arm by a threshold amount or a quantity. Other categorical simulation artifacts may be used, such as categorization based on predefined thresholds (e.g., based on a median survival time, a hazard ratio, a p-value, a percent survival after a predefined quantity of months). The categorial simulation artifacts may provide a binary output, indicating a success or failure, or may provide three or more outputs corresponding to a range of possible outcomes.

### 2.90 Providing Editable UI Input Elements to the Interactive GUI

S290, which includes providing to, at least a second display section of the interactive GUI, a set of editable user interface input elements, may function to enable a user to control one or more aspects of the clinical trial intelligence service. The set of editable user interface input elements, when manipulated, may configure or reconfigure one or more of the set of simulation parameters thereby enabling a re-execution of a succeeding set of virtual simulations and a real-time adaptation of the one or more graphical simulation artifacts based on the re-execution of the succeeding set of virtual simulations. S290 may be performed, in some examples, by a clinical trial intelligence service 101 as described with reference to FIG. 1.

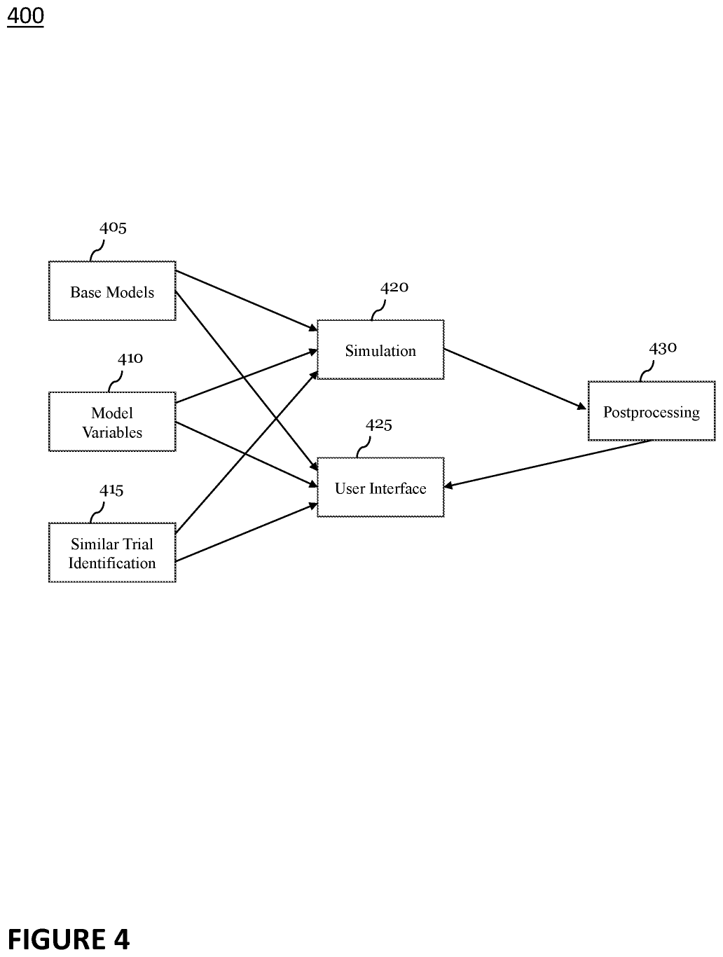

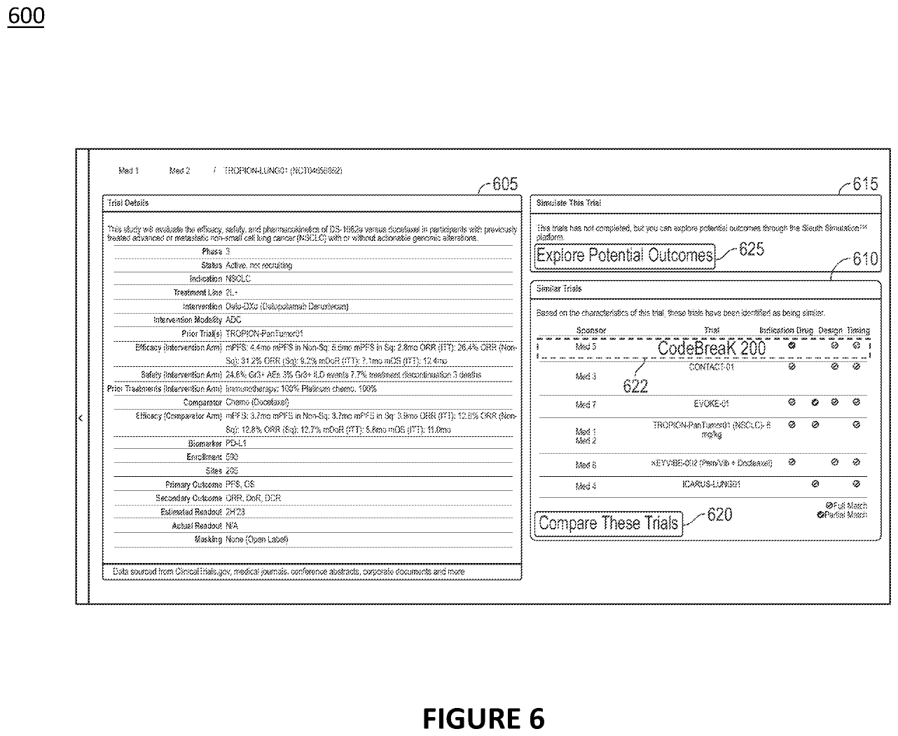

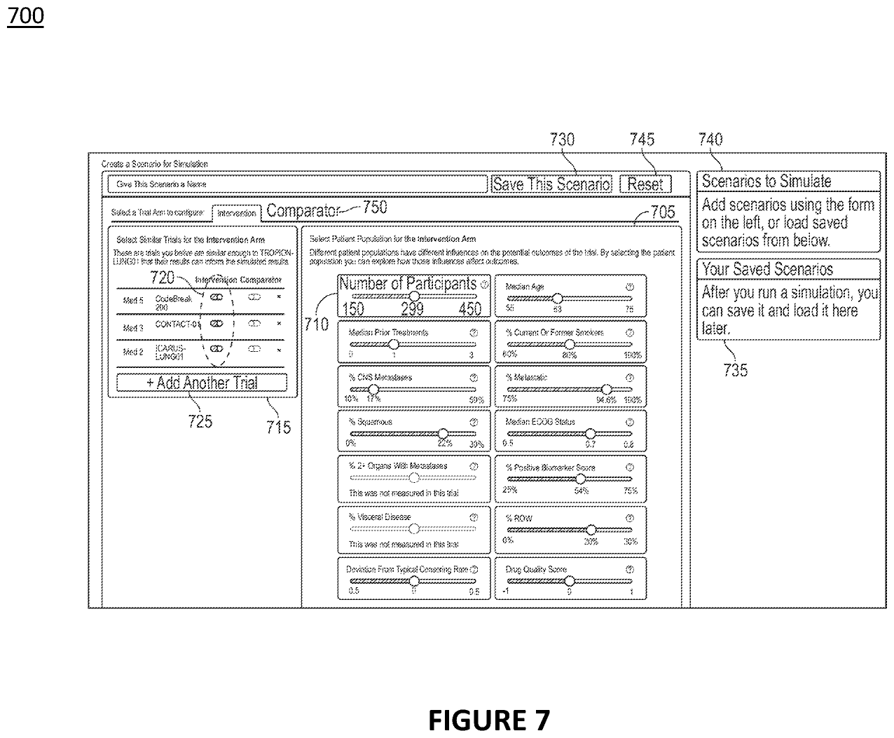

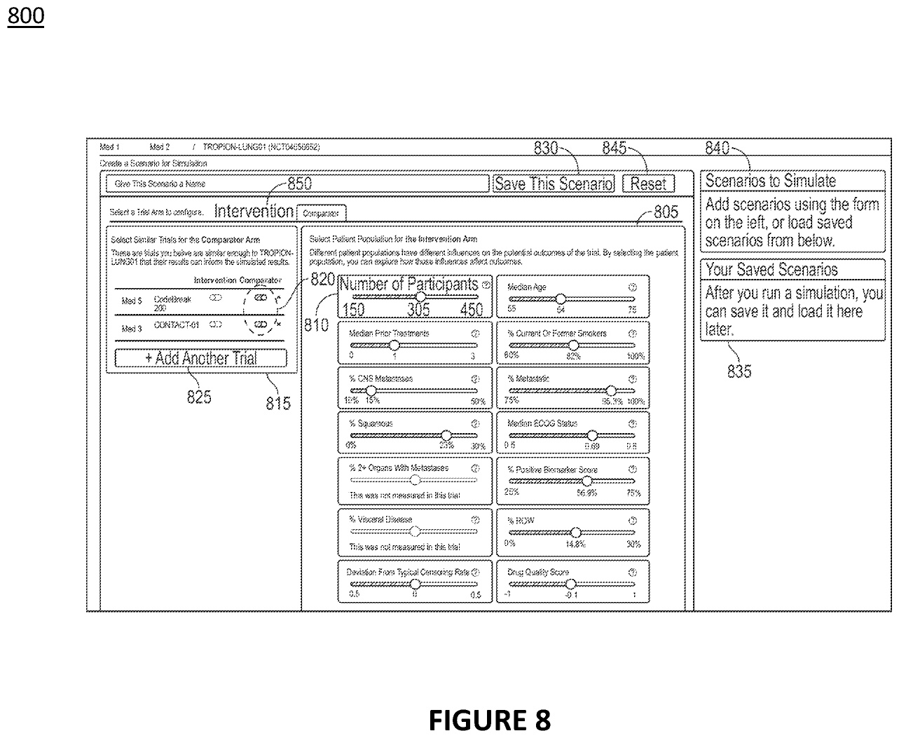

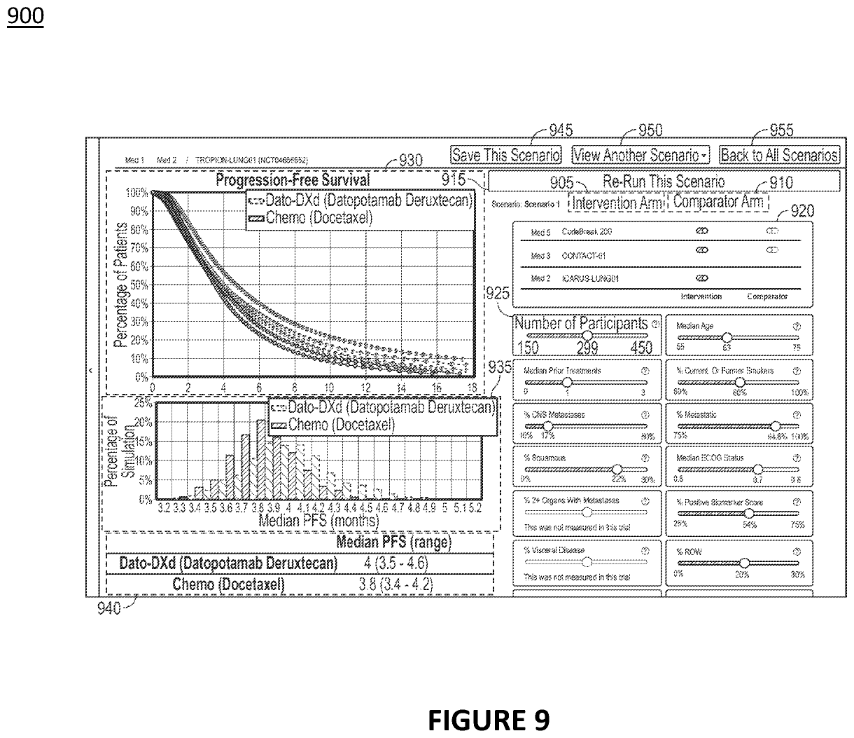

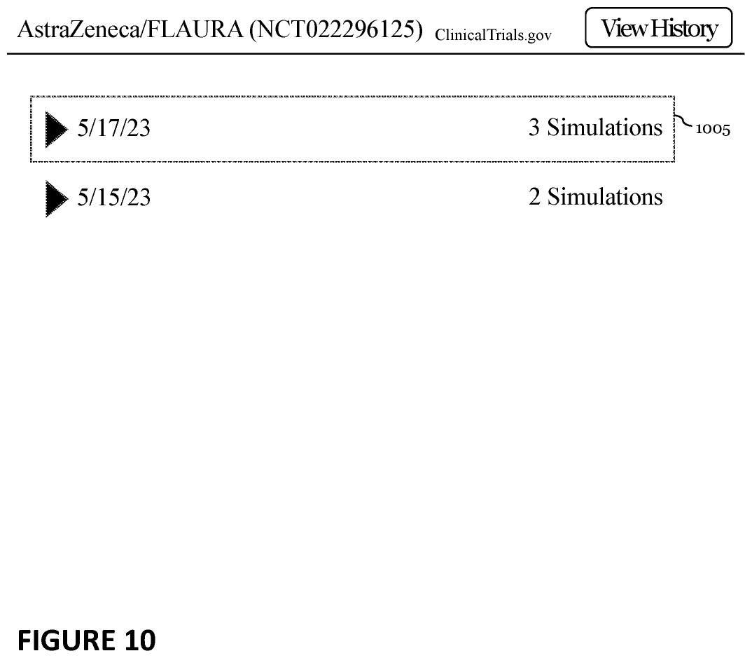

GUI views 600, 700, 800, 900, and 1000 of FIGS. 6 through 10 may represent various views of a GUI configured to enable a user to interact with a system performing method 200 (e.g., clinical trial intelligence service 101) via editable user interface input elements. In some examples, GUI views 600, 700, 800, 900, and 1000 may be examples of views of one or more of user interface 160 as described with reference to FIG. 1 or user interface 425 as described with reference to FIG. 4.

GUI view 600 may depict a view of UI elements related to a clinical trial. The GUI view may include a first UI component 605, a second UI component 610, and a third UI component 615. The first UI component 605 may provide information related to the clinical trial, which may be referred to as the target clinical trial. This information may be retrieved from a database (e.g., a database at clinical trial intelligence service 101 or in communication with clinical trial intelligence service 101) and may correspond to one or more sources (e.g., a source of source set 105).

The second UI component 610 may indicate a similarity of the clinical trial whose details are displayed in the first UI component 605 to other clinical trials stored at the database. The second UI component 610 may, in some examples, display similar clinical trials as a column of elements 622. Each element 622 within the column may have information corresponding to an identifier of a sponsor, an identifier of the trial name, and an indication of a level of similarity. In the present example, the level of similarity may be displayed according to which aspects of a similar clinical trial partially or fully match the clinical trial whose details are displayed by first UI component 605 (e.g., whether an indication parameter, a drug parameter, a design parameter, or a timing parameter fully or partially matches). In some examples, the indication of the level of similarity displayed in second UI component 610 may be determined according to the similarity algorithm described herein (e.g., according to the similarity ranking generated by clinical intelligence service 101). The second UI component 610 may additionally include a UI control component 620 configured to enable comparison of the clinical trial whose details are displayed in first UI component 605 with one or more of clinical trials displayed in second UI component 610.

The third UI component 615 may be configured to enable a user to simulate clinical trial outcomes (e.g., to execute virtual simulations) for the clinical trial whose details are displayed in first UI component 605. For instance, the third UI component 615 may include a UI control component 625 that may enable a user to simulate the clinical trial. Additionally, third UI component 615 may indicate, to a user, whether a simulation has been run for the clinical trial. In some examples, selecting UI control component 625 may generate GUI view 700 or GUI view 800.

GUI view 700 may depict a view of UI elements that a user may interact with to modify a simulation of an intervention clinical trial arm. The GUI view 700 may include a first UI component 705 and a second UI component 715. First UI component 705 may include a set of sliders that may correspond to one or more model variables associated with the intervention arm to be simulated. Each slider 710 of the set of sliders may correspond to a range of values for a particular model variable and may be configured to receive input from the user indicating a particular value within the range of values. The particular value selected by the user may be utilized when a simulation is performed for the intervention arm. In some examples, the set of sliders may be an example of a subset of the editable UI input elements described herein (e.g., the subset of the editable UI input elements used to adjust the distinct variable values of the target clinical trial for generating the synthetic statistical model).

The second UI component 715 may display a set of trials selected by a user that are similar to the intervention arm of the target clinical trial to be simulated. These selected trials may be displayed as elements of a column. The selected trials may be selectively toggled to have influence on the simulation for the intervention arm according to binary UI control elements 720. Additional trials may be added and displayed in the second UI component 715 via UI control component 725. In some examples, binary UI control elements 720 and UI control component 725 may be examples of a subset of the editable UI input elements described herein (e.g., the subset of the editable UI input elements used to add or remove candidate clinical trials).

GUI view 700 may further include UI control components 730 and 745. UI control component 730 may be configured to save a particular simulation scenario using a user-assigned name and UI control component 745 may be configured to refresh GUI view 700 to a default view. The default view may be defined as a view in which the first UI component 705 and second component 715 display default values.

GUI view 700 may additionally include third UI component 735 and fourth UI component 740. Third UI component 735 may be configured to display a list of simulation scenarios that have already been run and that may be loaded into fourth UI component 740. Fourth UI component 740 may be configured to display scenarios to simulate, either added via user interaction with third UI component 735 or via user interaction with control component 730. Selecting a scenario within UI component 740 may generate GUI view 900.

GUI view 800 may depict a view of UI elements that a user may interact with to modify a simulation of a control clinical trial arm, which may also be referred to as a “comparator clinical trial arm” or “comparator trial arm”. The GUI view may include a first UI component 805 and a second UI component 815. First UI component 805 may include a set of sliders that may correspond to one or more model variables associated with the control arm to be simulated. Each slider 810 of the set of sliders may correspond to a range of values for a particular model variable and may be configured to receive input from the user indicating a particular value within the range of values. The particular value selected by the user may be utilized when a simulation is performed for the control arm. In some examples, the set of sliders may be an example of a subset of the editable UI input elements described herein (e.g., the subset of the editable UI input elements used to adjust the distinct variable values of the target clinical trial for generating the synthetic statistical model).

The second UI component 815 may display a set of trials selected by a user that are similar to the control arm to be simulated. These selected trials may be displayed as elements of a column. The selected trials may be selectively toggled to have influence on the simulation for the control arm according to binary UI control elements 820. Additional trials may be added and displayed in the second UI component 815 via UI control component 825. In some examples, binary UI control elements 820 and UI control component 825 may be examples of a subset of the editable UI input elements described herein (e.g., the subset of the editable UI input elements used to add or remove candidate clinical trials).

GUI view 800 may further include UI control components 830 and 845. UI control component 830 may be configured to save a particular simulation scenario using a user-assigned name and UI control component 845 may be configured to refresh GUI view 800 to a default view. The default view may be defined as a view in which the first UI component 805 and second component 815 display default values.

GUI view 800 may additionally include third UI component 835 and fourth UI component 840. Third UI component 835 may be configured to display a list of simulation scenarios that have already been run and that may be loaded into fourth UI component 840. Fourth UI component 840 may be configured to display scenarios to simulate, either added via user interaction with third UI component 835 or via user interaction with control component 830. Selecting a scenario within UI component 840 may generate GUI view 900.

In some examples, a user may switch from GUI view 700 to GUI view 800 via selection of UI control element 750. Additionally, or alternatively, a user may switch from GUI view 800 to GUI view 700 via selection of UI control element 850. In some such examples, third UI component 735 and fourth UI component 740 may display the same information between views (e.g., third UI component 735 and third UI component 835 may display the same information when switching between views and fourth UI component 740 and fourth UI component 840 may display the same information when switching between views).

GUI view 900 may depict a view of UI elements that a user may interact with to see the results of a simulation (e.g., to view graphical simulation artifacts, numerical simulation artifacts, and categorial simulation artifacts) and to modify aspects of the control arm and/or the intervention arm for performing the simulation again. The GUI view 900 may include a UI component 930 displaying a graphical representation of a simulation result (e.g., a Kaplan-Meier curve of progression-free survival). The GUI view may include a UI component 935 displaying another graphical representation of the simulation result (e.g., a visualization of median progression-free survival dependent on a percentage of total simulations run for the control arm and the intervention arm). In some examples, each of UI component 930 and UI component 935 may be examples of graphical simulation artifacts. The GUI view may further include a UI component 940 displaying a numerical representation of the simulation result (e.g., a visualization of a median PFS for the control arm and the intervention arm along with an associated range, such as a confidence interval). In some examples, UI component 940 may be an example of a numerical simulation artifact.

The GUI view 900 may include a first UI control element 905 for the intervention arm and a second UI control element 910 for the comparator arm. Selecting the first UI control element 905 may generate a UI component 920 and a set of sliders 925 corresponding to the intervention arm and selecting the second UI control element 910 may generate a UI component 920 and a set of sliders 925 corresponding to the control arm. The UI component 920 may display clinical trials that are being factored into performing the simulation and may, in some examples, correspond to similar clinical trials selected previously at UI component 715 or UI component 815. The set of sliders 925 may display values of one or more model variables and may, in some examples, correspond to values selected previously at UI component 705 or UI component 805. Modifying the UI component 920 or the set of sliders 925 may cause the simulation to be run again and may result in UI components 930, 935, and 940 being updated (e.g., in real-time). In some examples, the set of sliders 925 may be an example of a subset of the editable UI input elements described herein (e.g., the subset of the editable UI input elements used to adjust the distinct variable values of the target clinical trial for generating the synthetic statistical model).

The GUI view 900 may further include UI control components 945, 950, and 955. UI control component 945 may be configured to enable a user to save the simulation scenario depicted in GUI view 900. UI control component 950 may be configured to enable a user to view another simulation scenario (e.g., a previously saved simulation scenario or a new simulation scenario). UI control component 955 may generate another GUI view that displays some or each saved scenario that the user may select between.

GUI view 1000 may depict a view of previously saved simulations that a user may select among. The simulations may be arranged in entries 1005 of a column arranged in a particular order (e.g., in order of a date when a simulation was run). Selecting one of the simulations may result in generating an associated GUI view for that simulation (e.g., GUI view 900). In some examples, the saved simulations may be saved in a database (e.g., a database at clinical trial prediction system 105 or in communication with clinical trial prediction system 105).

### 3. Computer-Implemented Method and Computer Program Product

The system and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processors and/or the controllers. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

Although omitted for conciseness, the preferred embodiments include every combination and permutation of the implementations of the systems and methods described herein.

As a person skilled in the art will recognize from the previous detailed description and from the figures and claims, modifications and changes can be made to the preferred embodiments of the invention without departing from the scope of this invention defined in the following claims.

What is claimed is:

1. A computer-implemented method comprising:
at a clinical intelligence service:
obtaining, via a computer network from one or more sources of digital data, a corpus of historical clinical trial data associated with each of a plurality of candidate clinical trials;
deriving, by one or more computer processors, a statistical model for each of the plurality of candidate clinical trials based on the corpus of historical clinical trial data associated with each of the plurality of candidate clinical trials;
computing, by the one or more computer processors, a composite statistical model based on calculating a weighted average of a combination of the statistical model for each of the plurality of candidate clinical trials;
generating, by the one or more computer processors, a synthetic statistical model for a target clinical trial based on adapting the composite statistical model using a plurality of distinct variable values extracted from data associated with a configuration of the target clinical trial;
initializing, by the one or more computers executing a virtual simulation computing system with a plurality of simulation parameters comprising a representation of the synthetic statistical model and a set of input values for a plurality of variables associated with the synthetic statistical model;
executing, by the virtual simulation computing system, a plurality of virtual simulations based at least on the initialization of the virtual simulation computing system with the plurality of simulation parameters;
generating, by the one or more computer processors, clinical trial intelligence data based on the execution of the plurality of virtual simulations, wherein the clinical trial intelligence data includes one or more graphical simulation artifacts; and
providing to:
at least a first display section of an interactive simulation graphical user interface (GUI), the one or more graphical simulation artifacts, and
at least a second display section of the interactive simulation GUI a set of editable user interface input elements that, when manipulated, configure or reconfigure one or more of the plurality of simulation parameters thereby enabling a re-execution of a succeeding plurality of virtual simulations and a real-time adaptation of the one or more graphical simulation artifacts based on the re-execution of the succeeding plurality of virtual simulations.

2. The computer-implemented method of claim 1, wherein calculating the weighted average comprises:
setting a respective weight for each candidate trial of the plurality of candidate clinical trials based at least in part on a population size associated with the candidate trial or a similarity of one or more variables associated with the candidate trial to the plurality of distinct variable values extracted from the data associated with the configuration of the target clinical trial, wherein computing the composite statistical model comprises:
applying each respective weight against a respective statistical model for the candidate trial to generate a weighted statistical model; and
combining each weighted statistical model to generate the composite statistical model.

3. The computer-implemented method of claim 2, wherein the one or more variables each comprise a characteristic of a population, a characteristic of a drug, a design characteristic of a clinical trial, or a characteristic of a disease.

4. The computer-implemented method of claim 1, further comprising the clinical intelligence service:
identifying the plurality of candidate clinical trials, wherein identifying the plurality of candidate clinical trials comprises utilizing a machine learning model, the machine learning model performing the steps of:
providing the corpus of historical clinical trial data to the machine learning model, the machine learning model specifically trained to infer confidence values for clinical trials corresponding to a level of similarity between the clinical trials and the target clinical trial; and
applying the confidence values to a minimum confidence value threshold to identify the plurality of candidate clinical trials.

5. The computer-implemented method of claim 1, wherein deriving the statistical model for each of the plurality of candidate clinical trials comprises:
extracting, from the corpus of historical clinical trial data, outcome data for a plurality of samples in the candidate clinical trial, wherein the outcome data comprises an outcome for each sample in the plurality of samples; and
executing, by the one or more computer processors, a regression on the set of samples to generate a probability distribution for the candidate clinical trial corresponding to the outcome data.

6. The computer-implemented method of claim 1, wherein generating the synthetic statistical model comprises:
extracting the plurality of distinct variable values from the data associated with the configuration of the target clinical trial data; and
transforming the composite statistical model into the synthetic statistical model based at least in part on applying the plurality of distinct variable values to the composite statistical model.

7. The computer-implemented method of claim 1, wherein initializing the virtual simulation computing system with the plurality of simulation parameters comprises:
loading the representation of the synthetic statistical model and the set of input values for the plurality of variables associated with the synthetic statistical model into a memory of the virtual simulation computing system, wherein the representation of the synthetic statistical model and the set of input values for the plurality of variables associated with the synthetic statistical mode are retrieved from the memory for executing the plurality of virtual simulations.

8. The computer-implemented method of claim 1, wherein the set of editable user interface input elements configure or reconfigure the one or more of the plurality of simulation parameters by triggering the clinical intelligence service to add a candidate clinical trial to the plurality of candidate clinical trials or to remove a candidate clinical trial for re-computation of the composite statistical model prior to re-execution of the succeeding plurality of virtual simulations.

9. The computer-implemented method of claim 1, wherein the set of editable user interface input elements configure or reconfigure the one or more of the plurality of simulation parameters by triggering the clinical intelligence service to update at least one of the plurality of distinct variable values associated with the configuration of the target clinical trial for re-generating the synthetic statistical model prior to re-execution of the succeeding plurality of virtual simulations.

10. The computer-implemented method of claim 1, wherein the plurality of simulation parameters comprises the plurality of candidate trials, wherein the set of editable user interface input elements configure the plurality of simulation parameters via selection of the plurality of candidate trials, and wherein initializing the virtual simulation computing system with the plurality of simulation parameters comprises:
loading the selected plurality of candidates into a memory of the virtual simulation computing system, wherein the selected plurality of candidates is retrieved from the memory for computing or re-computing the composite statistical model.

11. The computer-implemented method of claim 1, further comprising the clinical intelligence service:
obtaining, via the computer network from one or more second sources of digital data, a second corpus of historical clinical trial data associated with a second plurality of candidate clinical trials;
deriving a statistical model for each of the second plurality of candidate clinical trials based on the second corpus of historical clinical trial data associated with each of the second plurality of candidate clinical trials;
computing a second composite statistical model based on calculating a weighted average of a combination of the statistical model for each of the second plurality of candidate clinical trials;
generating, by the one or more computer processors, a second synthetic statistical model for the target clinical trial based on adapting the second composite statistical model using a second plurality of distinct variable values extracted from the data associated with the configuration of the target clinical trial;
initializing the virtual simulation computing system with a second plurality of simulation parameters comprising a representation of the second synthetic statistical model and a set of input values for a plurality of variables associated with the second synthetic statistical model; and
executing, by the virtual simulation computing system, a second plurality of virtual simulations based at least on the initialization of the virtual simulation computing system with the second plurality of simulation parameters, wherein the one or more graphical simulation artifact of the generated clinical trial intelligence data is based at least in part on executing the second plurality of virtual simulations.

12. The method of claim 11, wherein the plurality of simulation parameters corresponds to an intervention trial and the second plurality of simulation parameters corresponds to a comparator trial.

13. The computer-implemented method of claim 1, wherein the plurality of distinct variable values each comprise a population size, a characteristic of a drug, a design characteristic of a clinical trial, or a characteristic of a disease.

14. A computer-program product comprising a non-transitory machine-readable storage medium storing computer instructions that, when executed by one or more computer processors, perform operations comprising:
obtaining, at a clinical intelligence service via a computer network from one or more sources of digital data, a corpus of historical clinical trial data associated with each of a plurality of candidate clinical trials;
deriving a statistical model for each of the plurality of candidate clinical trials based on the corpus of historical clinical trial data associated with each of the plurality of candidate clinical trials;
computing a composite statistical model based on calculating a weighted average of a combination of the statistical model for each of the plurality of candidate clinical trials;
generating, by the one or more computer processors, a synthetic statistical model for a target clinical trial based on adapting the composite statistical model using a plurality of distinct variable values extracted from data associated with a configuration of the target clinical trial;
initializing a virtual simulation computing system with a plurality of simulation parameters comprising a representation of the synthetic statistical model and a set of input values for a plurality of variables associated with the synthetic statistical model;
executing, by the virtual simulation computing system, a plurality of virtual simulations based at least on the initialization of the virtual simulation computing system with the plurality of simulation parameters;
generating, by the one or more computer processors, clinical trial intelligence data based on the execution of the plurality of virtual simulations, wherein the clinical trial intelligence data includes one or more graphical simulation artifacts; and
providing to:
at least a first display section of an interactive simulation graphical user interface (GUI), the one or more graphical simulation artifacts, and
at least a second display section of the interactive simulation GUI a set of editable user interface input elements that, when manipulated, configure or reconfigure one or more of the plurality of simulation parameters thereby enabling a re-execution of a succeeding plurality of virtual simulations and a real-time adaptation of the one or more graphical simulation artifacts based on the re-execution of the succeeding plurality of virtual simulations.

15. The computer-program product of claim 14, wherein the operations to calculate the weighted average comprise:
setting a respective weight for each candidate trial of the plurality of candidate clinical trials based at least in part on a population size associated with the candidate trial or a similarity of one or more variables associated with the candidate trial to the plurality of distinct variable values extracted from the data associated with the configuration of the target clinical trial, wherein the operations to compute the composite statistical model comprise:
applying each respective weight against a respective statistical model for the candidate trial to generate a weighted statistical model; and
combining each weighted statistical model to generate the composite statistical model.

16. The computer-program product of claim 15, wherein the one or more variables each comprise a characteristic of a population, a characteristic of a drug, a design characteristic of a clinical trial, or a characteristic of a disease.

17. The computer-program product of claim 14, wherein the operations further comprise:
identifying the plurality of candidate clinical trials, wherein identifying the plurality of candidate clinical trials comprises utilizing a machine learning model, the machine learning model performing the steps of:
providing the corpus of historical clinical trial data to the machine learning model, the machine learning model specifically trained to infer confidence values for clinical trials corresponding to a level of similarity between the clinical trials and the target clinical trial; and
applying the confidence values to a minimum confidence value threshold to identify the plurality of candidate clinical trials.

18. A computer-implemented system comprising:
one or more processors;
a memory; and
a computer-readable medium operably coupled to the one or more computer processors, the computer-readable medium having computer-readable instructions stored thereon that, when executed by the one or more computer processors, cause a computing device to perform operations comprising:
obtaining, via a computer network from one or more sources of digital data, a corpus of historical clinical trial data associated with each of a plurality of candidate clinical trials;
deriving a statistical model for each of the plurality of candidate clinical trials based on the corpus of historical clinical trial data associated with each of the plurality of candidate clinical trials;
computing a composite statistical model based on calculating a weighted average of a combination of the statistical model for each of the plurality of candidate clinical trials;
generating, by the one or more computer processors, a synthetic statistical model for a target clinical trial based on adapting the composite statistical model using a plurality of distinct variable values extracted from data associated with a configuration of the target clinical trial;
initializing a virtual simulation computing system with a plurality of simulation parameters comprising a representation of the synthetic statistical model and a set of input values for a plurality of variables associated with the synthetic statistical model;
executing, by the virtual simulation computing system, a plurality of virtual simulations based at least on the initialization of the virtual simulation computing system with the plurality of simulation parameters;
generating, by the one or more computer processors, clinical trial intelligence data based on the execution of the plurality of virtual simulations, wherein the clinical trial intelligence data includes one or more graphical simulation artifacts; and
providing to:
at least a first display section of an interactive simulation graphical user interface (GUI), the one or more graphical simulation artifacts, and
at least a second display section of the interactive simulation GUI a set of editable user interface input elements that, when manipulated, configure or reconfigure one or more of the plurality of simulation parameters thereby enabling a re-execution of a succeeding plurality of virtual simulations and a real-time adaptation of the one or more graphical simulation artifacts based on the re-execution of the succeeding plurality of virtual simulations.

19. The computer-implemented system of claim 18, wherein the operations to calculate the weighted average comprise:
setting a respective weight for each candidate trial of the plurality of candidate clinical trials based at least in part on a population size associated with the candidate trial or a similarity of one or more variables associated with the candidate trial to the plurality of distinct variable values extracted from the data associated with the configuration of the target clinical trial, wherein the operations to compute the composite statistical model comprise:
applying each respective weight against a respective statistical model for the candidate trial to generate a weighted statistical model; and
combining each weighted statistical model to generate the composite statistical model.

20. The computer-implemented system of claim 19, wherein the one or more variables each comprise a characteristic of a population, a characteristic of a drug, a design characteristic of a clinical trial, or a characteristic of a disease.
